SEQUENCE LISTING (5 sequences)

SEQ ID NO: 1 - DNA, 372 bp, Saccharomyces cerevisiae
atggtcaaattaacttcaatcgctgccggtgttgccgccatcgctgccggtgttgccgctgctccagccactaccactctatctccatctgacgaaagagtcaacttggtcgaattgggtgtttacgtctccgatatcagagctcatttggctcaatactacttgtttcaagcagctcatccaactgagacctacccagttgagattgctgaagctgttttcaactatggtgacttcaccaccatgttgactggtattccagctgaacaagtcaccagagtcatcactggtgtcccatggtactccactagattgagaccagctatttccagtgctctatctaaggacggtatctacactgctattccaaaa

SEQ ID NO: 2 - protein, 124 aa, Saccharomyces cerevisiae
MVKLTSIAAGVAAIAAGVAAAPATTTLSPSDERVNLVELGVYVSDIRAHLAQYYLFQAAHPTETYPVEIAEAVFNYGDFTTMLTGIPAEQVTRVITGVPWYSTRLRPAISSALSKDGIYTAIPK

SEQ ID NO: 3 - DNA, 1001 bp, Saccharomyces cerevisiae
aatcaagaaatgcttacaactctactaacactgcagtttttcagctttctgattaatctcttcggtttaaattttttagcagctattactttctagtaacccttataacgcggttggtatcaaatcttcctgatagtacaagcagacctttgaccccattcttgttgtgtcttgaaatctttttttctgacaaaagtaagaagttacaccataatagagaagttaaataatgaaagtgtatcgatcatagaaatgatttcatttttttcaactactgttacggtcaagaaactaataatgaggataagttttcgcaacaatgattttggtgcaagtgatttaaagtcttgaattacttcaacacaatttgctttgcgacggtttagaataacgtaatcgtatcagcgaatccactaggcgcgcgtaaaaattacagactctaacaatatacgactcccagcacatatctactattctgtacaattccgggaaatagagggtacagtattattttttctcggcgatcgtttagggtatactggagataggcttagcaatacgcttctgaacgaggatctatagcaactaaattcagacattctgctacaccaaaaatggaagtatcacaggaacggaaaagggttttataaggcacctcggcacccctataactggcattctcacattcggcgcatacgaatagacaaaggccccagaaaacaataccttcgcatacagaatctataaagatgcatgcctcgtttaaaaacaagaacatcttcatagtgatcatgaaattgtgtgaaagatgagatatgtaatgcgtgaggtactaaatgaggatatgttaacgaatcgtttaagtggtgatgaccaatgaaggaataggtataaatagagatacttcaactatatgcctttgagaatatgtctttattccttcccttcctgttaagcttatatcagcactaacaaacaaaacaaatacaatggtcaaatta

SEQ ID NO: 4 - DNA, 13 bp, Artificial
  Description of Artificial Sequence supF-stuffer fragment junction component
ccccggagacgtc

SEQ ID NO: 5 - DNA, 10 bp, Artificial
  Description of Artificial Sequence terminal of supF gene
tcccccacca

(19) United States
(12) Patent Application Publication
(10) Pub. No.: US 2008/0050720 A1
(43) Pub. Date: Feb. 28, 2008

(54) Methods for Using a Sterol Biosynthesis Pathway Reporter Gene to Screen for Antifungal or Lipid Lowering Compounds

(76) Inventor: John W. Phillips, Pottersville, NJ (US)

Correspondence Address: ROSETTA INPHARMATICS, INC., 12040 - 115TH AVE. NE, KIRKLAND, WA, 98034, US

(21) Appl. No.: 10/566,426

(22) PCT Filed: Jul. 26, 2004
(86) PCT No.: PCT/US04/24034

§ 371 Date: Jan. 30, 2006

Related U.S. Application Data
(60) Provisional application No. 60/491,442, filed on Jul. 30, 2003.

(51) Int. Cl. C12Q 1/68

(52) U.S. Cl. 435/6

(57) ABSTRACT

The present invention relates to methods of using nucleotide sequences from the promoter region of a S. cerevisiae gene YRM325W whose expression is an indicator of inhibition or modulation of the sterol biosynthesis pathway in S. cerevisiae. The invention envisions using a target polynucleotide sequence, wherein the target polynucleotide sequence is operably linked to the promoter region of the YMR325W gene, to screen chemical libraries and natural products for compounds which can be used either as antifungal agents for use against a variety of fungal pathogens, or as lipid lowering agents to treat hypercholesterolemia. The invention also envisions using the methods of the invention to assay the efficacy of and/or specificity of antifungal agents and lipid lowering agents, and/or to monitor the activity of the sterol biosynthesis pathway.

### 1.0 INTRODUCTION

The present invention relates to methods of using nucleotide sequences from the promoter region a S. cerevisiae gene whose expression is an indicator of the inhibition or modulation of the sterol biosynthesis pathway in S. cerevisiae. The invention envisions using a target polynucleotide sequence, wherein the polynucleotide sequence is operably linked to the promoter region of the YMR325W gene, to screen chemical libraries and natural products for molecules which can be used as antifungal agents for use against a variety of fungal pathogens, or as lipid lowering agents to treat hypercholesterolemia. The invention also includes using the methods of the invention to assay the efficacy of and/or specificity of antifungal agents and lipid lowering agents, and/or to monitor the activity of the sterol biosynthesis pathway.

### 2.0 BACKGROUND OF THE INVENTION

Citation of a reference herein shall not be construed as an admission that such reference is prior art to the present invention.

2.1 Antifungal Agents

Fungi are eukaryotic microorganisms comprising a phylogenetic kingdom. The Kingdom Fungi is estimated to contain over 100,000 species and includes species of “yeast”, which is the common term for several families of unicellular fungi.

Although fungal infections were once unrecognized as a significant cause of disease, the extensive spread of fungal infections is a major concern in hospitals, health departments and research laboratories. According to a 1988 study, nearly 40% of all deaths from hospital-acquired infections were caused by fungi, not bacteria or viruses (Sternberg, S., 1994, Science 266:1632-34).

Immunocompromised patients are particularly at risk for fungal infections. Patients with impaired immune systems due to AIDS, cancer chemotherapy, or those treated with immunosuppressive drugs used to prevent rejection in organ transplant, are common hosts for fungal infections. Organisms including but not limited to Cryptococcus spp., Candida spp., Histoplasma spp., Coccidioides spp., and as many as 150 species of fungi have been linked to human or animal diseases (Sternberg, S., 1994, Science 266:1632-34). Under immunocompromised conditions, fungi that are normally harmless to the host when maintained in the gastrointestinal system, can be transferred to the bloodstream, eyes, brain, heart, kidneys, and other tissues leading to symptoms ranging in severity from white patches on the tongue, to fever, rupturing of the retina, blindness, pneumonia, heart failure, shock, or sudden catastrophic clotting of the blood (Sternberg, S., 1994, Science 266:1632-34). In susceptible burn victims, even S. cerevisiae (baker's yeast), common in the human mouth and normally non-virulent, can lead to severe infection (Sternberg, S., 1994, Science 266:1632-34). Hospital transmission may also occur via catheters or other invasive equipment (Sternberg, S., 1994, Science 266:1632-34).

Fungal infections are not limited to individuals with compromised immune systems. Geological and meterological events have been reported to trigger fungal outbreaks. Following a 1994 earthquake in California, tremors were estimated to have released infectious fungal spores from the soil triggering a 3-year statewide epidemic that led to more than 4,500 cases per year (Sternberg, S., 1994, Science 266:1632-34).

Moreover, fungal infections are not limited to humans. Animals and plants are both struck by fungal infections. The worldwide contamination of foods and feeds with mycotoxins, the secondary metabolites of fungi, is a significant problem that has adverse effects on humans, animals and crops and results in substantial illness and economic loss. (Hussein, H. S. and Brasel, J. M., 2001, Toxicology:167(2):101-34). The economic impact of mycotoxins includes loss of human and animal life, increased health care and veterinary care costs, reduced livestock production, disposal of contaminated foods and feeds, and investment in research and applications to reduce severity of the mycotoxin problem. (Hussein, H. S. and Brasel, J. M., 2001, Toxicology:167(2):101-34). Clearly, efforts to control the spread of fungi will concomitantly control the often costly byproducts of fungi, mycotoxins.

The widespread dissemination of fungal infection coupled with the recognition of fungi as a significant disease causing factor create an increasing need for antifungal agents. Existing antifungal therapies harbor many disadvantages, including the development of fungal resistance to known antifungal agents. The acquired resistance in fungal pathogens to known antifungal agents is likely to continue to fuel the search for novel and more effective antifungal agents.

An effective antifungal agent is toxic to the pathogenic fungi, but not to the host. One way to achieve both efficacy and safety to the host is to target a structure or pathway that is unique to the pathogen. For example, successful antibacterial therapies often take advantage of the differences between the prokaryotic bacteria and the eukaryotic host. However, since fungal pathogens, like human cells, are eukaryotic, it has been more difficult to identify therapeutic agents that uniquely affect the pathogen. A lack of sufficient pathogen specificity can result in host toxicity. Treatment of fungal diseases is often limited because antifungal agents are often toxic to the mammalian or plant host, frequently resulting in severe side effects. For example, the commonly prescribed drug, Amphotericin B, a mainstay of antifungal therapy, includes such side effects as fever, chills, low blood pressure, headache, nausea, vomiting, inflammation of blood vessels and kidney damage (Sternberg, S., 1994, Science 266:1632-34). Furthermore, many of the existing therapies act to inhibit or slow fungal growth, but do not kill the infecting fungi.

Currently, there are five main classes of antifungal compounds: flucytosine, candins, polyenes, allylamines, and azoles. Each class is characterized by its mode and/or site of action. Three of the five classes of effective antifuingal agents target sterol. Polyenes bind to fungal membrane sterol, resulting in the formation of aqueous pores through which essential cytoplasmic materials leak out; allylamines block ergosterol biosynthesis, leading to accumulation of squalene, which is toxic to cells; and azoles inhibit the synthesis of ergosterol, the main fungal sterol. (Balkis, M. M., et al., 2002, Drugs 62(7):1025-40). Therefore at present, there is a need in the art for efficient and economical methods to evaluate potential antifungal molecules for their effect on the sterol biosynthesis pathway.

2.2 Lipid Lowering Agents

Isoprenoids are a class of compounds involved in diverse cellular functions, including cell growth and sterol biosynthesis. Mevalonic acid is the precursor of isoprenoids, and cholesterol, a lipoprotein, is a product of the mevalonate pathway. Within cells, the concentration of mevalonate is tightly controlled through the activity of 3-hydroxy-3-methylglutaryl-CoA reductase (HMGCR), the enzyme that catalyzes the reduction of HMG-CoA to mevalonate.

Elevated cholesterol levels are a primary risk factor for coronary artery disease. This disease is a major problem in developed countries and currently affects 13 to 14 million adults in the United States alone. Dietary changes and drug therapy reduce serum cholesterol levels and dramatically decrease the risk of stroke and overall mortality (Eisenberg, 1998 Am. J. Med. 104:2S-5S). As shown in large-scale clinical trials, inhibition of HMGCR significantly decreases cholesterol levels and reduces the risks of stroke by 29% and the overall mortality by 22% (Hebert et al., 1997 J. Am. Med. Assoc. 278:313-21).

Inhibitors of HMGCR, commonly referred to as statins, are effective and safe drugs that are widely prescribed in cholesterol-lowering therapy. Statins curtail cholesterol biosynthesis by inhibiting the committed step in the biosynthesis of isoprenoids and sterols (Corsini, et al., 1995 Pharmacol. Res. 31:9-27). In general, statins are very effective in lowering serum cholesterol levels and are prescribed widely in treatment of hypercholesterolemia (Gotto, 1997 Am. J. Cardiol. 79,1663-6.

The evidence linking elevated serum cholesterol to coronary heart disease is overwhelming. For example, atherosclerosis is a slowly progressive disease characterized by the accumulation of cholesterol within the arterial wall. Compelling evidence supports the concept that lipids deposited in atherosclerotic lesions are derived primarily from plasma low-density lipoprotein (LDL); thus, LDLs have popularly become known as the “bad” cholesterol. In contrast, high-density lipoprotein (HDL) serum levels correlate inversely with coronary heart disease—indeed, high serum levels of HDL are regarded as a negative risk factor. It is hypothesized that high levels of plasma HDL are not only protective against coronary artery disease, but may actually induce regression of atherosclerotic plaques (e.g., see Badimon, et al., 1992 Circulation 86 (Suppl. III):86-94). Thus, HDL has popularly become known as “good” cholesterol. Inhibitors of HMGCR achieve clinical efficacy by a number of effects, including depletion of critical intracellular pools of sterols, and primarily inhibition of hepatic cholesterol biosynthesis.

The effectiveness of statin drugs in lowering cholesterol levels by inhibiting the synthesis of sterols is evidence that genes in the sterol biosynthesis pathway are good targets for lipid lowering drug therapy. Therefore at present, there is a need in the art for efficient and economical methods to evaluate potential lipid lowering molecules for their effect on the sterol biosynthesis pathway.

2.3 Monitoring the Sterol Biosynthesis Pathway

Sterol biosynthesis is accomplished via a complex pathway with multiple levels of feedback inhibition. It is therefore difficult to understand the role played by any one regulatory step in isolation. (Dimster-Denk, et. al., 1999 Journal of Lipid Research 40:850-60). Presently, individual targets are screened one at a time against drug compounds. The current invention provides the ability to screen for inhibition of all members of the pathway in one assay. The invention will enable the user to interrogate a compound library or collection of natural products and identify the subset of the library that blocks sterol biosynthesis regardless of the molecular target within the pathway.

Within the past decade, several technologies have made it possible to monitor the expression level of a large number of transcripts at any one time (see, e.g., Schena et al., 1995, Quantitative monitoring of gene expression patterns with a complementary DNA micro-array, Science 270:467-470; Lockhart et al., 1996, Expression monitoring by hybridization to high-density oligonucleotide arrays, Nature Biotechnology 14:1675-1680; Blanchard et al., 1996, Sequence to array: Probing the genome's secrets, Nature Biotechnology 14, 1649; U.S. Pat. No. 5,569,588, issued Oct. 29, 1996 to Ashby et al. entitled “Methods for Drug Screening”). In organisms for which the complete genome is known, it is possible to analyze the transcripts of all genes within the cell. With other organisms, such as humans, for which there is an increasing knowledge of the genome, it is possible to simultaneously monitor large numbers of the genes within the cell.

Such monitoring technologies have been applied to the identification of genes which are up-regulated or down-regulated in various diseased or physiological states, the analyses of members of signaling cellular states, and the identification of targets for various drugs. See, e.g., Friend and Hartwell, International Publication WO98/38329 (dated Sep. 3, 1998); Stoughton and Friend, U.S. Pat. No. 5,965,352 (issued on Oct. 12, 1999); Friend and Hartwell, U.S. Pat. Nos. 6,165,709 (issued on Dec. 26, 2000), 6,324,479 (issued on Nov. 27, 2001), all incorporated herein by reference for all purposes.

Levels of various constituents of a cell are known to change in response to drug treatments and other perturbations of the cell's biological state. Measurements of a plurality of such “cellular constituents” therefore contain a wealth of information about the effect of perturbations and their effect on the cell's biological state. Such measurements typically comprise measurements of gene expression levels of the type discussed above, but may also include levels of other cellular components such as, but by no means limited to, levels of protein abundances, or protein activity levels. The collection of such measurements is generally referred to as the “profile” of the cell's biological state.

The number of genes in a S. cerevisiae cell is typically on the order of more than 6,000 genes. The profile of a particular cell is therefore typically of high complexity. Any one perturbing agent may cause a small or a large number of cellular constituents to change their abundances or activity levels. Thus, identifying the particular cellular constituents which are associated with a certain biological pathway, such as the sterol biosynthesis pathway, provides a difficult and challenging task.

In order to efficiently monitor and study a particular biological pathway, it is necessary to have a “read-out” or reporter of the pathway which allows measurement of an alteration of the pathway. Many biological pathways, however, do not have reliable reporters associated with them. Therefore, there is a need in the art to identify reporter genes that are associated with a particular biological pathway. The present invention provides one such reporter gene and methods of using the inventive reporter to monitor the state of the sterol biosynthesis pathway in S. cerevisiae and additionally, methods of using the inventive reporter gene to screen chemical libraries and natural products for novel antifungal or lipid lowering agents.

### 3.0 SUMMARY OF THE INVENTION

The present invention relates to methods of using nucleotide sequences from the promoter region of a S. cerevisiae gene whose expression is an indicator of the inhibition or modulation of the sterol biosynthesis pathway in S. cerevisiae. This invention envisions using a target polynucleotide sequence, wherein the target polynucleotide sequence is operably linked to the promoter region of the YMR325W gene, to screen chemical libraries and natural products for molecules which can be used as either antifungal agents for use against a variety of fungal pathogens, or as lipid lowering agents to treat hypercholesterolemia. This invention also envisions using the methods of the invention to assay the efficacy of and/or specificity of antifungal agents and lipid lowering agents, and/or to monitor the activity of the sterol biosynthesis pathway.

One aspect of the invention provides a method for determining whether a molecule affects the function or activity of a sterol biosynthesis pathway in a S. cerevisiae cell comprising: (a) contacting the cell with, or recombinantly expressing within the cell, the molecule; (b) determining whether the RNA expression or protein expression in the cell corresponding to a target polynucleotide sequence is changed in step (a) relative to the expression of the target polynucleotide sequence in the absence of the molecule, the the target polynucleotide sequence being regulated by a promoter native to the gene YMR325W, and homologs thereof; and (c) determining that the molecule affects the function or activity of the sterol biosynthesis pathway if the expression is changed, or determining that the molecule does not affect the function or activity of the sterol biosynthesis pathway if the expression is unchanged.

In a particular embodiment, the invention further comprises the step of determining that the molecule inhibits sterol biosynthesis if a cell contacted with the molecule exhibits a lower level of sterol than a cell which is not contacted with the molecule. In another embodiment, the step of determining whether the RNA expression or protein expression has changed comprises determining whether RNA expression is changed. In still another embodiment, the step of determining whether the RNA expression or protein expression has changed comprises determining whether protein expression is changed. In another embodiment, the step of determining whether the molecule inhibits sterol biosynthesis comprises determining that the molecule inhibits sterol biosynthesis if the expression of the target polynucleotide sequence in step (a) is increased relative to the expression of the target polynucleotide sequence in the absence of the molecule.

In another embodiment, the S. cerevisiae cell is a cell that recombinantly expresses the target polynucleotide sequence. In a particular embodiment, wherein step (a) comprises contacting the cell with the molecule, step (a) is carried out in a liquid high throughput-like assay. In another embodiment, wherein step (a) comprises contacting the cell with the molecule, step (a) is carried out in a solid plate halo assay. In another embodiment, wherein step (a) comprises contacting the cell with the molecule, step (a) is carried out in an agar overlay assay.

In yet another embodiment, the cell comprises a promoter region of YMR325W, and homologs thereof, the promoter region being operably linked to a marker gene; and wherein step (b) comprises determining whether the RNA expression or protein expression of the marker gene is changed in step (a) relative to the expression of the marker gene in the absence of the molecule. In a preferred embodiment, the marker gene is selected from the group consisting of green fluorescent protein, red fluorescent protein, blue fluorescent protein, luciferase, LEU2, LYS2, ADE2, TRP1, CAN1, CYH2, GUS, CUP1 and chloramphenicol acetyl transferase.

Another aspect of the invention provides a method for determining the effect of a molecule upon the function or activity of the sterol biosynthesis pathway comprising: (a) contacting a S. cerevisiae cell with, or recombinantly expressing within the cell the molecule; (b) detecting a change in RNA expression or protein expression in the cell of a target polynucleotide sequence relative to the expression of the target polynucleotide sequence in the absence of the molecule, each target polynucleotide sequence being regulated by a promoter native to the gene YMR325W and homologs thereof; and (c) determining the effect of the molecule upon the function or activity of the sterol biosynthesis pathway based upon the change in RNA expression or protein expression. In one embodiment, step (a) comprises contacting the cell with said molecule. In another embodiment, step (a) comprises recombinantly expressing within the cell the molecule. In yet another embodiment, step (b) comprises detecting an increase in the RNA or protein expression, and step (c) comprises determining that the effect of the molecule is to inhibit the function or activity of the sterol biosynthesis pathway.

Another aspect of the invention provides a method for monitoring the activity of the sterol biosynthesis pathway in a S. cerevisiae cell exposed to a molecule comprising (a) contacting the cell with, or recombinantly expressing within the cell, the molecule; (b) determining whether the RNA expression or protein expression in the cell of a target polynucleotide sequence is changed in step (a) relative to the expression of the target polynucleotide sequence in the absence of the molecule, the target polynucleotide sequence being regulated by a promoter native to the gene YMR325W and homologs thereof; and (c) determining that the activity of the sterol biosynthesis pathway in the cell is changed if the expression is determined to be changed in step (b), or determining that the activity of the sterol biosynthesis pathway in the cell is unchanged if the expression is determined to be unchanged in step (b). In one embodiment of the invention, step (a) comprises contacting the cell with the molecule. In another embodiment of the invention, step (a) comprises recombinantly expressing within the cell the molecule. In yet another embodiment of the invention, step (b) comprises determining that expression is increased, and step (c) comprises determining that the activity of the sterol biosynthesis pathway is inhibited.

Another aspect of the invention provides a method for identifying a molecule that modulates the expression of a sterol biosynthesis pathway target polynucleotide sequence comprising: (a) recombinantly expressing in a S. cerevisiae cell, or contacting a S. cerevisiae cell with, at least one candidate molecule; and (b) measuring the RNA or protein expression in the cell of a target polynucleotide sequence, the target polynucleotide sequence being regulated by a promoter native to the gene YMR325W, and homologs thereof, wherein an increase or decrease in the expression of the target polynucleotide sequence relative to the expression of the target polynucleotide sequence in the absence of the candidate molecule indicates that the molecule modulates expression of the sterol biosynthesis pathway target polynucleotide sequence.

Yet another aspect of the invention provides a method for determining whether a first S. cerevisiae cell is mutant for a sterol biosynthesis pathway gene comprising: (a) in the first S. cerevisiae cell, determining the RNA or protein expression of a target polynucleotide sequence, the target polynucleotide sequence being regulated by a promoter native to the gene YMR325W and homologs thereof, wherein the cell is not being exposed to an inhibitor of the sterol biosynthesis pathway; (b) determining whether the RNA and/or protein expression of the target polynucleotide sequence determined in step (a) is changed relative to the RNA and/or protein expression of the target polynucleotide sequence in a second S. cerevisiae cell which is believed to have functional sterol biosynthesis genes; and (c) determining that the first S. cerevisiae cell is mutant for a sterol biosynthesis pathway gene if the expression is determined to be changed in step (a), or determining that the first S. cerevisiae cell is not mutant for a sterol biosynthesis pathway gene if the expression is determined to be unchanged in step (b). In another embodiment, the invention further comprises determining the RNA or protein expression of YMR325W, and homologs thereof, in the first S. cerevisiae cell; and wherein step (c) further comprises determining that the first S. cerevisiae cell is mutant for the sterol biosynthesis pathway gene if the expression of YMR325W is determined to be unchanged.

### 4.0 DETAILED DESCRIPTION OF THE INVENTION

The present invention relates to methods of using nucleotide sequences from the promoter region of a S. cerevisiae gene whose expression is an indicator of the inhibition or modulation of the sterol biosynthesis pathway in S. cerevisiae. This invention envisions using a target polynucleotide sequence, wherein the target polynucleotide sequence is operably linked to the promoter region of the YMR325W gene, to screen chemical libraries and natural products for molecules that can be used either as antifungal agents for use against a variety of fungal pathogens, or as lipid lowering agents to treat hypercholesterolemia. This invention also envisions using the methods of the invention to assay the efficacy of and/or specificity of antifungal agents and lipid lowering agents, and/or to monitor the activity of the sterol biosynthesis pathway.

As used herein, a reporter gene for the sterol biosynthesis pathway is the YMR325W gene for which a change in expression of its encoded RNA or protein is indicative of a change in the activity of the sterol biosynthesis pathway. Thus, the reporter gene of this invention is useful for analyzing the activity of the sterol biosynthesis pathway, e.g., to identify potential lipid lowering molecules and, or antifungal molecules which inhibit or modulate the sterol biosynthesis pathway.

In a preferred embodiment, the cell used in the methods of the invention is a S. cerevisiae cell. A preferred S. cerevisiae strain is one for which the genomic sequence is known, such as strain S288C or substantially isogeneic derivatives of it (see, e.g., Dujon et al., 1994, Nature 369:371-378; Bussey et al., 1995, Proc. Natl. Acad Sci. U.S.A. 92:3809-3813; Feldmann et al., 1994, E.M.B.O. J. 13:5795-5809; Johnston et al., 1994, Science 265:2077-2082; Galibert et al., 1996, E.M.B.O. J. 15:2031-2049). However, other strains may be used as well. S. cerevisiae strains are available, e.g., from American Type Culture Collection, 10801 University Boulevard, Manassas, Va. 20110-2209. Well-established methods are available for controllably modifying expression of S. cerevisiae genes. Standard techniques for manipulating S. cerevisiae are described in C. Kaiser, S. Michaelis, & A. Mitchell, 1994, Methods in Yeast Genetics: A Cold Spring Harbor Laboratory Course Manual, Cold Spring Harbor Laboratory Press, New York; and Sherman et al., 1986, Methods in Yeast Genetics: A Laboratory Manual, Cold Spring Harbor Laboratory, Cold Spring Harbor, N.Y. Many other strains commonly known and available in the art can be used.

Although for simplicity the disclosure often makes reference to single cells (e.g., “RNA is isolated from a cell exposed to a particular drug”), it will be understood by those of skill in the art that more often than not, any particular step of the invention will be carried out using a plurality of genetically similar cells, e.g., from a cultured cell line. Such similar cells are referred to herein as a “cell type.”

In accordance with the present invention there may be employed conventional molecular biology, biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel et al. (eds.), Current Protocols in Molecular Biology, John Wiley & Sons, Inc. (2002).

The invention is illustrated by way of examples set forth in Section 6 below which disclose, inter alia, the identification and characterization of a reporter gene of the S. cerevisiae sterol biosynthesis pathway, using Genome Reporter Matrix™ (“GRM”) technology (see U.S. Pat. Nos. 5,569,588, issued Oct. 29, 1996, and 5,777,888, issued Jul. 7, 1998, both of which are hereby incorporated by reference in their entireties).

For clarity of disclosure, and not by way of limitation, the detailed description of the invention is divided into the subsections which follow.

4.1 Sterol Biosynthesis Pathway Reporter Gene

The present invention relates to methods of using nucleotide sequences from a S. cerevisiae gene whose expression is an indicator of the inhibition or modulation of the sterol biosynthesis pathway in S. cerevisiae. The present invention identifies the gene YMR325W as a sterol biosynthesis pathway reporter gene. The YMR325W nucleotide sequences that are used in the present invention may comprise the entire YMR325W gene or fragments thereof, including, for example, the 5′ region of the YMR325W gene including the promoter, all or part of the coding region, or conservatively modified variants or homologs thereof that retain the indicator function of the YMR325W gene. A polynucleotide sequence corresponding to an open reading frame encoding a YMR325W protein is provided by SEQ ID NO: 1. An amino acid sequence for YMR325W is provided by SEQ ID NO: 2.

As used herein the term “promoter” refers to a nucleotide sequence that is necessary and sufficient in the presence of the appropriate factors to promote transcription of an operatively linked sequence. In preferred embodiments, the promoter of the YMR325W gene comprises, consists, or consists essentially of SEQ ID NO: 3, and homologs thereof. Homologs of SEQ ID NO: 3 may contain conservative substitutions, additions or deletions that do not effect the ability of the sequence to promote transcription of an operatively linked sequence. The ability of the YMR325W gene promoter sequence homolog to promote transcription of an operatively linked sequence may be tested by any method known in the art. One non-limiting method comprises linking a detectable marker gene such as green fluorescent protein (“GFP”) to a putative promoter sequence, detecting the transcription level of the marker gene, and comparing said transcription level to that produced in the absence of the promoter sequence. In some embodiments, the marker gene may be the YMR325W coding sequence. In this embodiment, activation or inhibition of the sterol biosynthesis pathway reporter gene in response to an agent or stimuli is determined by measuring changes in the level of YMR325W transcription.

Specific embodiments of the invention provide methods for using the promoter region of the YMR325W gene or locus as a reporter for the sterol biosynthesis pathway. In these embodiments, the promoter region of the YMR325W gene may be operably linked to a marker gene encoding a detectable or selectable product such as, but not limited to, GFP or an RNA transcript. Detection or selection of the marker RNA or protein is used to determine the activation or inhibition of the sterol biosynthesis pathway reporter gene in response to controlled stimuli.

4.2 Methods for Obtaining Sterol Biosynthesis Pathway Reporter Genes

The YMR325W gene or promoter region thereof can be isolated from any source, preferably from a S. cerevisiae cell or genomic library. Methods for obtaining genes are well known in the art, as described in Sambrook et al., 1989, supra.

Alternatively, the YMR325W gene or promoter region can be obtained by chemical synthesis, by cDNA cloning, or by the cloning of genomic DNA, or fragments thereof, purified from the desired cell (See, for example, Sambrook et al., 1989, supra; Glover, D. M. (ed.), 1985, DNA Cloning: A Practical Approach, MRL Press, Ltd., Oxford, U.K. Vol. I, II). Clones derived from genomic DNA may contain regulatory and intron DNA regions in addition to coding regions; clones derived from cDNA will not contain intron sequences.

Any S. cerevisiae cell having a functional YMR325W gene can serve as the nucleic acid source for the molecular cloning of the YMR325W gene or promoter region. The DNA may be obtained by standard procedures known in the art from cloned DNA (e.g., a DNA “library”), including EST libraries and cDNA libraries prepared from cells with high level expression of the protein.

Identification of a specific DNA fragment containing a YMR325W sterol biosynthesis pathway reporter gene or promoter region can be accomplished by various methods known in the art. For example, a portion of the YMR325W gene exemplified below can be purified and labeled to prepare a labeled probe, and the generated DNA may be screened by nucleic acid hybridization to the labeled probe (Benton and Davis, Science 196:180, 1977; Grunstein and Hogness, Proc. Natl. Acad. Sci. U.S.A. 72:3961, 1975). Those DNA fragments with substantial homology to the probe, such as an allelic variant, will hybridize. In a specific embodiment, high stringency hybridization conditions are used to identify an allelic variant of the YMR325W gene.

YMR325W gene sequences can also be obtained, e.g., by polymerase chain reaction (“PCR”) amplification of genomic DNA or cloned sequences. PCR primers are preferably chosen based on the YMR325W polynucleotide sequences described herein [SEQ ID NO: 1, SEQ ID NO: 2]. Computer programs that are well known in the art are useful in the design of primers with the required specificity and optimal amplification properties, such as Oligo version 5.0 (National Biosciences). PCR methods are well known in the art, and are described, for example, in Innis et al., eds., 1990, PCR Protocols: A Guide to Methods and Applications, Academic Press Inc., San Diego, Calif.

An alternative means for generating the nucleotide sequences of the invention is by synthesis of synthetic polynucleotides or oligonucleotides, e.g., using N-phosphonate or phosphoramidite chemistries (Froehler et al., 1986, Nucleic Acid Res. 14:5399-5407; McBride et al., 1983, Tetrahedron Lett. 24:246-248).

A YMR325W gene derivative can be made by altering encoding nucleotide sequences by substitutions, additions or deletions that provide for functionally equivalent molecules. Alternatively, non-functional mutant forms of the sterol biosynthesis pathway reporter protein, that may for example compete with the wild-type sterol biosynthesis pathway reporter protein in the sterol biosynthesis pathway, but which are less effective, can be prepared for use in screening potential antifungal molecules.

Due to the degeneracy of nucleotide coding sequences, other DNA sequences which encode substantially the same amino acid sequence as the YMR325W gene may be used in the practice of the present invention. These include but are not limited to allelic genes and nucleotide sequences comprising all or portions of sterol biosynthesis pathway reporter genes which are altered by the substitution of different codons that encode the same amino acid residue within the sequence, thus producing a silent change.

The nucleotide sequences encoding sterol biosynthesis pathway reporter gene promoter regions, derivatives and analogs of the invention can be produced by various methods known in the art. The manipulations which result in their production can occur at the gene or protein level. For example, a cloned sterol biosynthesis pathway reporter gene sequence can be modified by any of numerous strategies known in the art (Sambrook et al., 1989, supra). The sequence can be cleaved at appropriate sites with restriction endonuclease(s), followed by further enzymatic modification if desired, isolated, and ligated in vitro. In the production of a derivative or analog of the YMR325W gene, care should be taken to ensure that the modified gene remains within the same translational reading frame as the gene, uninterrupted by translational stop signals, in the gene region where the desired activity is encoded.

Additionally, the YMR325W gene can be mutated in vitro or in vivo, to create and/or destroy translation, initiation, and/or termination sequences, or to create variations in coding regions and/or form new restriction endonuclease sites or destroy preexisting ones, to facilitate further in vitro modification. Any technique for mutagenesis known in the art can be used, including but not limited to, in vitro site-directed mutagenesis (Hutchinson, C., et al., 1978, J. Biol. Chem. 253:6551; Zoller and Smith, 1984, DNA 3:479-488; Oliphant et al., 1986, Gene 44:177; Hutchinson et al., 1986, Proc. Natl. Acad. Sci. U.S.A. 83:710), etc. PCR techniques are preferred for site directed mutagenesis (see Higuchi, 1989, “Using PCR to Engineer DNA”, in PCR Technology: Principles and Applications for DNA Amplification, H. Erlich, ed., Stockton Press, Chapter 6, pp. 61-70).

4.3 Methods for Measuring Sterol Biosynthesis Pathway Reporter Gene Expression

This invention provides several methods for detecting changes in gene expression or protein expression, including but not limited to the expression of the YMR325W gene, and marker genes operably linked to the YMR325W reporter gene of the invention. Assays for changes in gene expression are well known in the art (see e.g., PCT Publication No. WO 96/34099, published Oct. 31, 1996, which is incorporated by reference herein in its entirety). Such assays may be performed in vitro using transformed cell lines, immortalized cell lines, or recombinant cell lines.

The RNA expression or protein expression of a target polynucleotide sequence, regulated by a promoter native to the YMR325W gene may be measured by measuring the amount or abundance of RNA (as RNA or cDNA) or protein. The target polynucleotide sequence may be, but is not limited to, a marker gene or the YMR325W gene coding region. For example, the target polynucleotide may be an untranslated region of a gene. In a specific embodiment, the target polynucleotide sequence is an open reading frame. In a preferred embodiment, the target polynucleotide sequence is a marker gene. In particular, the assays may detect the presence of increased or decreased expression of a target polynucleotide sequence on the basis of increased or decreased mRNA expression (using, e.g., nucleic acid probes), increased or decreased levels of protein products (using, e.g., antibodies thereto), or increased or decreased levels of expression of a marker gene (e.g., GFP) operably linked to the YMR325W 5′ promoter region in a recombinant construct.

The present invention envisions monitoring changes in sterol biosynthesis pathway reporter gene expression or marker gene expression by any expression analysis technique known to one of skill in the art, including but not limited to, differential display, serial analysis of gene expression (SAGE), nucleic acid array technology, oligonucleotide array technology, microarray expression analysis, reverse-transcription polymerase chain reaction (RT-PCR), dot blot hybridization, northern blot hybridization, subtractive hybridization, protein chip arrays, Western blot, immunoprecipitation followed by SDS PAGE, immunocytochemistry, proteome analysis and mass-spectrometry of two-dimensional protein gels.

Methods of gene expression profiling to measure changes in gene expression are well-known in the art, as exemplified by the following references describing RT-PCR (Bachmair et al., 2002, Methods Mol. Biol. 193:103-116; Muller et al., 2002, Biotechniques, 32(6):1372-4, 1376, 1378-9), subtractive hybridization (Wang and Brown, 1991, Proc. Natl. Acad. Sci. U.S.A. 88:11505-11509), differential display (Liang and Pardee, 1992, Science 257:967-971), SAGE (Velculescu et al., 1995, Science 270:484-487), proteome analysis (Humphery-Smith et al., 1997, Electrophoresis 18:1217-1242; Dainese et al., 1997, Electrophoresis 18:432-442), and hybridization-based methods employing nucleic acid arrays (Heller et al., 1997, Proc. Natl. Acad. Sci. U.S.A. 94:2150-2155; Lashkari et al., 1997, Proc. Natl Acad. Sci. U.S.A. 94:13057-13062; Wodicka et al., 1997, Nature Biotechnol. 15:1259-1267). Microarray technology is described in more detail below.

In one series of embodiments, various expression analysis techniques may be used to identify molecules that affect sterol biosynthesis pathway reporter gene expression or marker gene expression, by comparing a cell line expressing the YMR325W gene or marker gene under the control of the YMR325W gene promoter sequence in the absence of a test molecule to a cell line expressing the same sterol biosynthesis pathway reporter gene or marker gene under the control of the YMR325W gene promoter sequence in the presence of the test molecule. In a preferred embodiment, expression analysis techniques are used to identify a molecule which upregulates sterol biosynthesis pathway reporter gene or marker gene expression upon treatment of a cell with the molecule.

In a specific embodiment, nucleic acid array technology (preferably small arrays, e.g., arrays having less than about 1,000 hybridization probes, or more preferably having less than about 100 hybridization probes) may be used to determine the YMR325W gene or marker gene expression pattern in a S. cerevisiae cell not exposed to a test molecule for comparison with the YMR325W gene or marker gene expression pattern of a S. cerevisiae cell exposed to a test molecule. In a preferred embodiment, a protocol similar to the one described in Gene Cloning and Expression Technologies, 2002, eds. Weiner and Lu, BioTechniques Press, Chpt. 36 is utilized.
4.3.1 Preferred Methods for Monitoring Reporter Gene Expression of the YMR325W Gene

### Heterologous Sterol Biosynthesis Pathway Reporter Gene Construct

In a one embodiment, the S. cerevisiae cell being assayed for sterol biosynthesis pathway reporter gene expression contains a fusion construct of the YMR325W gene transcriptional promoter region operably linked to a marker gene expressing a detectable and/or selectable product. In a preferred embodiment, the promoter of the YMR325W gene comprises, consists, or consists essentially of SEQ ID NO: 3. In one embodiment, the detectable or selectable product is a protein. In another embodiment, the detectable product is a RNA. Increased expression of a marker gene operably linked to the YMR325W gene promoter indicates increased expression of the YMR325W gene.

The marker gene is a sequence encoding a detectable or selectable marker, the expression of which is regulated by the YMR325W gene promoter region in the heterologous construct used in the present invention. In one embodiment, the detectable or selectable marker is a protein. In another embodiment, the detectable marker is a RNA. Preferably, the assay is carried out in the absence of background levels of marker gene expression (e.g., in a cell that is mutant or otherwise lacking in the marker gene). If not already lacking in endogenous marker gene activity, cells mutant in the marker gene may be selected by known methods, or the cells can be made mutant in the marker gene by known gene-disruption methods prior to introducing the marker gene (Rothstein, 1983, Meth. Enzymol. 101:202-211).

A marker gene of the invention may be any gene which encodes a detectable and/or selectable product. The detectable marker may be any molecule that can give rise to a detectable signal, e.g., a fluorescent protein or a protein that can be readily visualized or that is recognizable by a specific antibody or that gives rise enzymatically to a signal. The selectable marker can be any molecule which can be selected for its expression, e.g., which gives cells a selective advantage over cells not having the selectable marker under appropriate (selective) conditions. In preferred aspects, the selectable marker is an essential nutrient of which the cell in which the interaction assay occurs is mutant or otherwise lacks or is deficient, and the selection medium lacks such nutrient. In one embodiment, one type of marker gene is used to detect gene expression. In another embodiment, more than one type of marker gene is used to detect gene expression.

Preferred marker genes include, but are not limited to, green fluorescent protein (Cubitt et al., 1995, Trends Biochem. Sci. 20:448-455), red fluorescent protein, blue fluorescent protein, luciferase, LEU2, LYS2, ADE2, TRP1, CAN1, CYH2, GUS, CUP1 or chloramphenicol acetyl transferase (CAT). Other marker genes include, but are not limited to, URA3, HIS3 and/or the lacZ genes (see e.g., Rose and Botstein, 1983, Meth. Enzymol. 101:167-180) operably linked to GAL4 DNA-binding domain recognition elements. Alam and Cook disclose non-limiting examples of detectable marker genes, which can be operably linked to the YMR325W gene promoter region (Alam and Cook, 1990, Anal. Biochem. 188:245-254).

In a specific embodiment of the invention, a marker gene is operably linked to the promoter of YMR325W. In an alternate embodiment, more than one different marker gene is used to detect transcriptional activation, e.g., one encoding a detectable marker, and one or more encoding one or more different selectable marker(s), or e.g., different detectable markers. Expression of the marker genes can be detected and/or selected for by techniques known in the art (see e.g. U.S. Pat. Nos. 6,057,101 and 6,083,693).

Methods to construct a suitable reporter construct are disclosed herein by way of illustration and not limitation and any other methods known in the art may also be used. In a preferred embodiment, the reporter gene construct is a chimeric reporter construct comprising a marker gene that is transcribed under the control of the YMR325W gene promoter sequence comprising all or a portion of a promoter region of YMR325W. If not already a part of the DNA sequence, the translation initiation codon, ATG, is provided in the correct reading frame upstream of the DNA sequence.

Vectors comprising all or portions of the gene sequences of YMR325W useful in the construction of recombinant S. cerevisiae reporter gene constructs and cells are provided. The vectors of this invention also include those vectors comprising DNA sequences which hybridize under stringent conditions to the YMR325W gene sequences, and conservatively modified variations thereof.

The vectors of this invention may be present in transformed or transfected cells, cell lysates, or in partially purified or substantially pure forms. DNA vectors may contain a means for amplifying the copy number of the gene of interest, stabilizing sequences, or alternatively may be designed to favor directed or non-directed integration into the host cell genome.

Given the strategies described herein, one of skill in the art can construct a variety of vectors and nucleic acid molecules comprising functionally equivalent nucleic acids. DNA cloning and sequencing methods are well known to those of skill in the art and are described in an assortment of laboratory manuals, including Sambrook et al., 1989, supra; and Ausubel et al., 2002 Supplement.

Transformation and other methods of introducing nucleic acids into a host cell (e.g., transfection, electroporation, liposome delivery, membrane fusion techniques, high velocity DNA-coated pellets, viral infection and protoplast fusion) can be accomplished by a variety of methods which are well known in the art (see, for instance, Ausubel, supra, and Sambrook, supra). S. cerevisiae cells of the invention can be transformed or transfected with an expression vector, such as a plasmid, a cosmid, or the like, wherein the expression vector comprises the DNA of interest. Alternatively, the cells may be infected by a viral expression vector comprising the DNA or RNA of interest.

Particular details of the transfection and expression of nucleotide sequences are well documented and are understood by those of skill in the art. Further details on the various technical aspects of each of the steps used in recombinant production of foreign genes in expression systems can be found in a number of texts and laboratory manuals in the art (see, e.g., Ausubel et al., 2002, herein incorporated by reference).

### Detecting Reporter Gene Expression

In accordance with the present invention, reporter gene expression can be monitored at the RNA or the protein level. In a specific embodiment, molecules which affect reporter gene expression may be identified by detecting differences in the level of marker protein expressed by S. cerevisiae cells contacted with a test molecule versus the level of marker protein expressed by S. cerevisiae cells in the absence of the test molecule.

Protein expression can be monitored using a variety of methods which are well known to those of skill in the art. For example, protein chips or protein microarrays (e.g., ProteinChip™, Ciphergen Biosystem) and two-dimensional electrophoresis (see e.g., U.S. Pat. No. 6,064,754 which is incorporated herein by reference in its entirety) can be utilized to monitor protein expression levels. As used herein “two-dimensional electrophoresis”) (2D-electrophoresis) means a technique comprising isoelectric focusing, followed by denaturing electrophoresis, generating a two-dimensional gel (2D-gel) containing a plurality of proteins. Any protocol for 2D-electrophoresis known to one of ordinary skill in the art can be used to analyze protein expression by the reporter genes of the invention. For example, 2D electrophoresis can be performed according to the methods described in O'Farrell, 1975, J. Biol. Chem. 250: 4007-4021.

### Liquid High Throughput-Like Assay

In a preferred embodiment, a liquid high throughput-like assay is used to determine the protein expression level of the YMR325W gene. The following exemplary, but not limiting, assay may be used:

A reporter construct is transformed into a wild-type S. cerevisiae strain, e.g., ABY12. Cultures from solid media plates are used to inoculate liquid cultures in Casamino Acids media or an equivalent media This liquid culture is grown and then diluted in Casamino Acids media or an equivalent media.

A test molecule is selected for the assay, preferably but not necessarily along with a negative control molecule. The test molecule and negative control molecule are separately added to an assay plate containing multiple wells and serially diluted (e.g., 1 to 2) into Casamino Acids media plus DMSO in sequential columns, so that each plate contains a range of concentrations of each drug. If a negative control is being used, one column of each plate may be used as a “no drug” control, containing only Casamino Acids media plus DMSO. The skilled artisan will note that different assay plates may be used, such as those with 96, 384 or 1536 well format.

An aliquot of liquid reporter strain is added to each well of the serial dilution plates from above and mixed. The assay plates are then incubated. In a preferred embodiment, they are incubated at 30° C. for 24 hours.

After incubation the assay plates are analyzed for detectable marker gene product. In a preferred embodiment, the assay plates are imaged in a Molecular Dynamics Fluorimager SI to measure the fluorescence from the GFP reporters.

The results are then analyzed, as described above. If the drug is an inhibitor of the sterol biosynthesis pathway, the specific sterol biosynthesis pathway reporters will show increases in fluorescence for the higher drug concentrations versus the lower drug concentrations and/or the no drug controls.

### Solid Plate Halo Assay

Additionally, the following exemplary, but not limiting, assay may be used to determine whether a test molecule inhibits the sterol biosynthesis pathway in S. cerevisiae. Although described for YMR325W, other sterol biosynthesis pathway reporter genes, and homologs thereof, may be used.

A YMR325W reporter construct is transformed into wild-type S. cerevisiae strain, such as ABY12. The transformed strain is grown on a solid Casamino Acids media or an equivalent media plate. The culture from the solid media plate is used to inoculate a liquid culture in (e.g., Casamino Acids) media. This liquid culture is grown and then diluted in Casamino Acids media or an equivalent media. Cell culture is then spread evenly over the surface of each of two or more solid agar-media plates to form a lawn of the YMR325W reporter strain on each plate.

Two blank paper discs are placed on top of the agar surface of each plate evenly spaced apart. In one embodiment, 6 mm diameter paper discs are used. (Becton Dickinson #231039). On one plate, an appropriate amount of the test molecule is spotted onto one of the two paper discs (low concentration treatment) and DMSO is spotted on the other paper disc as a control. On another plate a greater amount of the test molecule is spotted onto one of the two paper discs (high concentration treatment) and DMSO is spotted on the other paper disc as a control. The plates are then incubated.

After incubation, the assay plates are analyzed as described above. In a preferred embodiment, the assay plates are imaged in a Molecular Dynamics Fluorimager SI to measure the fluorescence from the GFP reporters. The results are then examined, an increase in sterol biosynthesis pathway reporter gene expression and a halo of no growth around the test molecule disc indicating inhibition of the sterol biosynthesis pathway and the potential utility of the test molecule as an antifingal agent.

### Agar Overlay Method

Agar overlays may be prepared by any method known in the art, including but not limited to the preparation methods described herein. An agar plate is prepared containing a layer of bacteria or fungi. An second layer, an agar overlay containing the YMR325W reporter gene strain, is placed over the first layer of agar. The plate is incubated and the YMR325W layer is then examined for any effects of the natural products produced by the first agar layer containing the bacteria or fungal natural products. In one embodiment, following incubation, the plate is sprayed with a tetrazolium salt (e.g., MTT) which is converted to a formazan dye by the microorganism, thereby revealing inhibition zones of little or no growth as clear spots against a purple background. In one embodiment, the first agar layer is a grid of test strains, whereas the second agar layer comprises a YMR325W reporter construct fusion strain. Any agar overlay method known to one of skill in the art may be modified and used in connection with the present invention including but not limited to those described in Rahalison, L. et al., 1991, Phytochem. Anal. 2: 199-203 and Rios et al., 1988, J. Ethnopharmacol. 23(2-3):12749, hereby incorporated by reference in their entireties.
4.3.2 Other Methods for Monitoring Reporter Gene Expression of the YMR325W gene

### Small Array Assays

Sterol biosynthesis pathway YMR325W reporter gene expression may be monitored on the nucleic acid level or the protein level using small arrays as described in Martel et al., Proc. SPIE Vol. 4626: 35-43, Biomedical Nanotechnology Architectures and Applications, D. Bornhop et al. eds., the contents of which are hereby incorporated by reference in its entirety. In a preferred embodiment, a multiplexed mRNA assay to measure the expression of 16 genes may be conducted as described below.

Array plates contain the same 16-element array at the bottom of each well. In a preferred embodiment, the plate contains 96 wells. Each array element consists of a unique target (“anchor”) polynucleotide sequence that incorporates a position-specific sequence. The binding specificity of the array elements may be modified to render them target-specific. This consists of a single hybridization step that modifies the binding specificity of the array elements. This is achieved using programming linker species. Each programming linker contains both an array element-binding oligonucleotide region and a target-specific region. The array is exposed to a mixture of programming linker species, each species hybridizes to its corresponding element in the array and presents its target-specific region at that position. If the target-specific region of the programming linker is also an oligonucleotide, then the array is capable of subsequently immobilizing other nucleic acids. If instead the target-specific region of the programming linker is an antibody, then the linker-modified array element exposes an antibody that can capture the corresponding protein antigen. One or more of the array elements are designed to report on the level of expression of the YMR325W reporter gene at the protein or transcript level.

### Genome Reporter Matrix™ Technology

One method of monitoring the expression of an YMR325W reporter gene are YMR325W promoter gene fusion constructs that are part of a Genome Reporter Matrix™ (“GRM”), or an equivalent thereof. The description below of the generation of gene expression profiles utilizing the Genome Reporter Matrix™ has been described essentially in U.S. Pat. Nos. 5,569,588, and 5,777,888, and Dimster-Denk, et al., 1999, J. Lipid Research, 40:850-860, all of which are incorporated herein by reference, in their entireties.

The promoter (and optionally, 5′ upstream regulatory elements and/or 5′ upstream untranslated sequences) of a S. cerevisiae sterol biosynthesis pathway reporter ORF or a S. cerevisiae sterol biosynthesis pathway reporter gene is fused to a marker gene creating a transcriptional and/or translational fusion of the promoter to the marker gene. The promoter and optional additional sequences comprise all the regulatory elements necessary for transcriptional (and optionally translational) control of an attached coding sequence. The marker gene is a detectable marker gene that can be any gene that, when expressed in a suitable host, encodes a product that can be detected by a quantitative assay. Any suitable assay may be used, including but not limited to enzymatic, calorimetric, fluorescence or other spectrographic assays, fluorescent activated cell sorting assay and immunological assays. Examples of suitable marker genes include, inter alia, green fluorescent protein, β-lactamase, lacZ, invertase, membrane bound proteins (e.g., CD2, CD4, CD8, the influenza hemagglutinin protein, and others well known in the art) to which high affinity antibodies directed to them exist or can be made routinely, fusion protein comprising membrane bound protein appropriately fused to an antigen tag domain (e.g., hemagglutinin or Myc and others well known in the art). In a preferred embodiment, the marker protein is GFP from the jellyfish Aequorea victoria. GFP is a naturally fluorescing protein that does not require the addition of any exogenous substrates for activity. The ability to measure GFP fluorescence in intact living cells makes it an ideal marker protein for the GRM or an equivalent matrix comprising living cells.

In a preferred embodiment, reporter constructs comprise the 5′ region of the ORF comprising the promoter of the ORF and other expression regulatory sequences, and generally, the first four codons of the ORF fused in-frame to the green fluorescent protein. In a more preferred embodiment, approximately 1000 base-pairs of 5′ regulatory sequence are included in each fusion. Only 228 S. cerevisiae ORFs (3.5%) possess introns. Of these 228 intron-containing ORFs, all but four contain only one intron. In these ORFs, fusions are created two to four codons past (3′ to) the splice junction. Therefore, these fusions must undergo splicing in order to create a functional reporter fusion.

Each reporter fusion is preferably assembled in an episomal yeast shuttle vector (either CEN or 2μ plasmid) or on a yeast integrating vector for subsequent insertion into the chromosomal DNA. In a preferred embodiment, the gene reporter constructs are built using a yeast multicopy vector. A multicopy vector is chosen to facilitate easy transfer of the reporter constructs to many different S. cerevisiae strain backgrounds. In addition, the vector replicates at an average of 10-20 copies per cell, providing added sensitivity for detecting genes that are expressed at a low level. In another preferred embodiment, the reporter constructs are maintained on episomal plasmids in S. cerevisiae.

In one embodiment, a plurality (all or a significant subset) of the resulting sterol biosynthesis pathway reporter gene constructs, including a YMR325W reporter gene construct, is transformed into a strain of S. cerevisiae. The resulting strains constitute one embodiment of the Genome Reporter Matrix™. In another embodiment, the Genome Reporter Matrix™ comprises reporter gene constructs for all or a significant subset of the open reading frames of the S. cerevisiae genome, including a YMR325W reporter gene construct.

Expression profiles can be produced by arraying wild-type or mutant cells carrying the reporter fusion genes in growth media containing one or more different drugs, chemical compounds, and/or known or potential antifungal molecules and measuring changes in expression of the marker gene by the appropriate assay (see below). As used herein a “wild-type” cell is understood to be any strain or isolate of cells having a designated genotype or phenotype and is used in an experiment as the reference or control strain. In contrast, a “mutant” cell is one that has a genotype or phenotype that is different from a wild-type cell due to the presence of a mutation in the genetic information of the mutant cell. In a preferred embodiment, where the marker gene is GFP, measurement of changes in expression are done by measuring the amount of green light produced by the cells over time with an automated fluorescence scanner. Alternatively, the drug(s), chemical compound(s), and/or known or potential antifungal molecule(s) may be added to the S. cerevisiae cells after they have been arrayed onto growth media and then measuring changes in marker gene expression by the appropriate assay. In another embodiment, the test molecules are recombinantly expressed in the S. cerevisiae cells.

In one embodiment, a natural product screen is used in the methods of the invention. In another embodiment, a direct bioautography method is used in the methods of the invention. In yet another embodiment, an agar overlay screening assay is used.

Over 93% of the reporter genes are detectable over background signals on rich medium. The reproducibility of individual reporter genes is high, with expression generally varying by less than 10%.

In one embodiment, the GRM, including a YMR325W reporter construct, is used to obtain gene expression information. The GRM is preferred to hybridization-based methods of profiling for several reasons. First, because the promoter-marker fusions include the first four amino acids of the native gene product, the response profiles are composites of both transcriptional and translational effects. The importance of being able to monitor both levels of response is underscored by the experience with bacterial antibiotics. Those antibiotics that work at the translational level have a greater therapeutic performance than those affecting transcription. Because hybridization-based methods can reveal only effects on transcription, profiling with the GRM provides a more complete view of the full spectrum of biological effects induced by exposure to drugs, compounds, and/or known or potential antifungal molecules.

Second, the GRM permits profiling of gene expression changes in living cells, which permits one to easily measure the kinetics of changes in gene response profiles in the same population of cells following exposure to different drugs and chemical agents.

Third, hybridization-based methods require relatively sophisticated molecular procedures to produce labeled cDNA, followed by a hybridization of labeled cDNA probes to target DNA arrays on slides or chips. The GRM requires only that being able to produce arrays of colonies and measure emitted light. These procedures are easier to scale up in an industrial setting than are sophisticated molecular biology methods, rendering data that is more straightforward to produce and more reproducible in nature.

### Microarray Technology

The invention herein provides methods of using microarray technology to assay sterol biosynthesis pathway reporter gene expression, in particular YMR325W gene expression. Microarrays may be prepared by any method known in the art, including but not limited to the preparation methods described herein below.

In one embodiment, hybridization levels are measured by microarrays of probes consisting of a solid phase on the surface of which are immobilized a population of polynucleotides, such as a population of DNA or DNA mimics, or, alternatively, a population of RNA or RNA mimics. In preferred embodiments, a microarray comprises a support or surface with an ordered array of binding (e.g., hybridization) sites or “probes” for products of one or more of the genes in the genome of a cell or organism, including the YMR325W sterol biosynthesis pathway reporter gene. The polynucleotide molecules which may be analyzed by the present invention are from S. cerevisiae cells containing a promoter region from the YMR325W gene. In one embodiment, the polynucleotide molecules analyzed by the invention comprise RNA, including, but by no means limited to, total cellular RNA, poly(A)⁺ messenger RNA (mRNA), fraction thereof, or RNA transcribed from cDNA (i.e., cRNA; see, e.g., Linsley & Schelter, U.S. Pat. No. 6,271,002).

It will be appreciated that when cDNA complementary to the RNA of a cell is made and hybridized to a microarray under suitable hybridization conditions, the level of hybridization to the site in the array corresponding to any particular gene will reflect the prevalence in the cell of mRNA transcribed from that gene.

In preferred embodiments, cDNAs from two different cells are hybridized to the binding sites of the microarray. In the case of drug responses, one cell is exposed to a test compound and another cell of the same type is not exposed to the test compound. The cDNA derived from each of the two cell types are differently labeled so that they can be distinguished. The relative abundance of an mRNA in two cells or cell lines is scored as perturbed (i.e., the abundance is different in the two sources of mRNA tested) or as not perturbed (i.e., the relative abundance is the same). It is, however, also advantageous to determine the magnitude of the relative difference in abundances for an mRNA in two cells or in two cell lines. This can be carried out, as noted above, by calculating the ratio of the emission of the two fluorophores used for differential labeling, or by analogous methods that will be readily apparent to those of skill in the art.

4.4 Molecules that May be Screened by the Methods of the Invention

This invention envisions using the YMR325W gene of the invention to screen chemical libraries and natural products for molecules which can be used either as antifingal agents against a variety of pathogenic fungal species, or as lipid lowering agents to treat hypercholesterolemia. This invention also envisions using the YMR325W gene of the invention to assay the efficacy of and/or specificity of antifungal agents and lipid lowering agents, and/or to monitor the activity of the sterol biosynthesis pathway.

Any molecule, e.g. protein or non-protein organic pharmaceutical, with the potential capability of affecting the YMR325W gene may be screened. In a preferred embodiment, a plurality of assay mixtures are run in parallel with different concentrations to obtain a differential response to the various concentrations. In another preferred embodiment, one of these concentrations serves as a negative control, i.e. at zero concentration or below the level of detection. This invention also envisions assaying the efficacy and/or specificity of antifungal agents and lipid lowering agents.

In one embodiment of the invention, test molecules are contacted with the sterol biosynthesis pathway reporter cells of the invention. In another embodiment, test molecules are recombinantly expressed in the sterol biosynthesis pathway YMR325W reporter cells.

Test molecules may be any of numerous chemical classes. In a specific embodiment, the test molecules are organic molecules, preferably small molecules, i.e., those having a molecular weight of more than 50 and less than about 2,500 daltons. In another specific embodiment, the test molecules comprise biomolecules including, but not limited to: peptides, saccharides, fatty acids, steroids, purines, pyrimidines, derivatives, structural analogs or combinations thereof.

The test molecules to be screened may be selected or derived from a wide variety of sources including libraries of synthetic and/or natural compounds. In a specific embodiment, the test molecules are purified compounds. In another embodiment, the test molecules are produced by an organism such as strains of bacteria or fungi, e.g., agar overlay assay. In a specific embodiment, the test molecules are produced by random and/or directed synthesis of one or more organic compounds, including but not limited to, expression of randomized oligonucleotides, oligopeptides and/or saccharides. Alternatively, libraries of natural compounds in the form of bacterial, fungal, plant and animal extracts are available from e.g. Pan Laboratories (Bothell, Wash.) or MycoSearch (NC), or are readily producible.

Synthetic compound libraries are commercially available from Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich (Milwaukee, Wis.).

Additionally, natural and synthetically produced libraries and compounds are readily modified through conventional chemical, physical, and biochemical means (Blondelle et al., Tib Tech, 14:60, 1996). In a specific embodiment, known compounds and/or known antifungal agents may be subjected to directed or random chemical modifications, such as acylation, alkylation, esterification, amidification, etc., to produce structural analogs. In another specific embodiment, test molecules may also be created using methods such as rational drug design or computer modelling.

In a specific embodiment of the invention, the natural products envisioned by the present invention are microorganisms and/or potential antifungal or lipid lowering compounds produced by microorganisms. The following non-limiting procedure may be used to isolate microorganisms and/or potential antifungal or lipid lowering compounds for use in the screening procedures described herein. The procedure described below was used to isolate the antifungal Ascosteroside, and is provided by way of example and not limitation (Gorman, J. A., et al., 1995, J. Antibiotics, 49(6): 547-552).

A sample of soil or other organic matter is collected and suspended in diluent (such as buffered saline), sonicated for several minutes and mixed. This initial suspension is then diluted and aliquots are plated onto different types of nutrient agar and incubated at room temperature. After several days, colonies are subcultured onto agar medium and incubated for several days at room temperature. Test molecules may be selected from the colonies and then screened by the methods described herein. Other methods known in the art for screening natural products are contemplated by the instant invention, including but not limited to those described in McCormack et al., 1994, Appl. Envir. Microbiology 60(3): 927-931 and Bojase et al., 2002, Planta Med. 68:615-620, both of which are hereby incorporated by reference in their entireties.

In another specific embodiment, known or potential antifungal agent(s) or lipid lowering agents serve as test molecules to determine the specificity and/or efficacy of the molecule. In a particular embodiment of the invention, known antifungal agents or known lipid lowering agents are tested for whether the antifungal agent or the lipid lowering agent affects the sterol biosynthesis pathway.

4.5 Pharmaceutical Applications

Molecules identified by the methods of the present invention as having e.g., antifungal activity, can be used to treat diseases and disorders caused by a fungus, e.g., fungal infections. The present invention envisions the use of molecules identified by the methods of the present invention against several fungal species including but not limited to the pathogenic fungal species disclosed in Section 2.0 of the specification, particularly those listed in Table 1 below.

TABLE 1
Fungal Species

| | | |
|---|---|---|
| Cryptococcus spp. | Geotrichum spp. | Exophiala spp. |
| Asperigillus spp. | Epidermophyton floccosum | Dematiaceous fungi |
| Coccidioides spp. | Sporothrix spp. | Fonsecaea pedrosoi |
| Blastomyces spp. | Microsporum spp. | Acremonium strictum |
| Sporothrix spp. | Scytalidium spp. | Cladophilophora |
| | | bantiana |
| Rhizopus spp. | Acremonium spp. | Pityrosporum spp. |
| Paeciliomyces spp. | Candida spp. | Epidermophyton spp. |
| Curvularia spp. | Histoplasma spp. | Malassezia spp. |
| Wangiella spp. | Paracoccidioides spp. | Trichophyton spp. |
| Pneumocystis | Fusarium spp. | Hendersonula |
| carninii | | toruloidea |
| Scedosporium spp. | Trichosporon spp. | Scopulariopsis |
| | | brevicaulis |
| Bipolaris spp. | Pseudallescheria spp. | Piedraia hortae |
| Phialophora spp. | Alternaria spp. | |

In particular embodiments, the molecules identified by the methods of the present invention are used against Candida albicans, Candida krusei, Candida lusitaniae, Candida parapsilosis, Candida glabrata, Aspergillus fumigatus, and Cryptococcus neoformans. The molecules identified by the methods of the present invention may be used to treat fungal infections in a variety of subjects including, but not limited to, humans, non-human animals, and crops, including, but not limited to, domestic and farm animals such as dogs, cats, chickens, bovids, goats, pigs, horses, fish, birds, silkworms, and plants such as corn, wheat, rice and tobacco.

Molecules identified by the methods of the present invention as having e.g. lipid lowering activity, can be used to treat diseases and disorders caused by e.g., high levels of LDL cholesterol. The present invention envisions the use of molecules identified by the methods of the present invention in the treatment of diseases and disorders that include, but are not limited to, vascular disease (in particular, artherosclerosis), lipid storage diseases, obesity, diabetes, hypercholesterolemia, cancer, and osteoporosis. Compounds that treat hypercholesterolemia are particularly important because of the cause-and-effect relationship between hypercholesterolemia and morbidity and mortality from coronary artery disease (CAD) (For a review, see Mahley, R. W. and Bersot, T. P., In, Goodman & Gilman's The Pharmacological Basis of Therapeutics, 10th Ed., McGraw-Hill, New York, 1996, Ch. 36, pp. 984-995).

The molecules identified by the methods of the present invention may also be tested in yeast cell systems and heterologous host cell systems (e.g., human cells) to verify that they do not have undesirable side effects. In addition, the GRM can be used to make sure that the compounds do not adversely alter gene transcription (e.g., in an undesirable way). Of course, certain changes in gene expression may be inevitable and many of these will not be deleterious to the patient or host organism. Once lead molecules have been identified, these molecules can be refined further via rational drug design and other standard pharmaceutical techniques.

The molecules of this invention may be formulated into pharmaceutical compositions and administered in vivo at an effective dose to treat a particular disease or condition. Determination of a preferred pharmaceutical formulation and a therapeutically efficient dose regiment for a given application is within the skill of the art taking into consideration, for example, the condition and weight of the patient, the extent of desired treatment and the tolerance of the patient for the treatment.

Administration of the molecules, including isolated and purified forms, their salts or pharmaceutically acceptable derivatives thereof, may be accomplished using any conventionally accepted mode of administration.

The pharmaceutical compositions of this invention may be in a variety of forms, which may be selected according to the preferred modes of administration. These include, for example, solid, semi-solid and liquid dosage forms such as tablets, pills, powders, liquid solutions or suspensions, suppositories, and injectable and infusible solutions. The preferred form depends on the intended mode of administration and therapeutic application. Modes of administration may include oral, parenteral, subcutaneous, intravenous, intralesional or topical administration.

The molecules of this invention may, for example, be placed into sterile, isotonic formulations with or without cofactors which stimulate uptake or stability. The formulation is preferably liquid, or may be lyophilized powder. For example, the inhibitors may be diluted with a formulation buffer comprising 5.0 mg/ml citric acid monohydrate, 2.7 mg/ml trisodium citrate, 41 mg/ml mannitol, 1 mg/ml glycine and 1 mg/ml polysorbate 20. This solution can be lyophilized, stored under refrigeration and reconstituted prior to administration with sterile Water-For-Injection (USP).

Topical administration includes administration to the skin or mucosa, including surfaces of the lung and eye. Compositions for topical administration, including those for inhalation, may be prepared as a dry powder which may be pressurized or non-pressurized. In non-pressurized powder compositions, the active ingredient in finely divided form may be used in admixture with a larger-sized pharmaceutically acceptable inert carrier comprising particles having a size, for example, of up to 100 micrometers in diameter. Alternatively, the composition may be pressurized and contain a compressed gas, such as nitrogen or a liquified gas propellant. The liquified propellant medium and indeed the total composition is preferably such that the active ingredient does not dissolve therein to any substantial extent.

Dosage forms for topical or transdermal administration of a molecule of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this invention.

The pharmaceutical compositions may also be administered using microspheres, microparticulate delivery systems or other sustained release formulations placed in, near, or otherwise in communication with affected tissues or the bloodstream. Suitable examples of sustained release carriers include semipermeable polymer matrices in the form of shaped articles such as suppositories or microcapsules. Implantable or microcapsular sustained release matrices include polylactides (U.S. Pat. No. 3,773,319; EP 58,481), copolymers of L-glutamic acid and gamma ethyl-L-glutamate (Sidman et al., 1985); poly(2-hydroxyethyl-methacrylate) or ethylene vinyl acetate (Langer et al., 1981, Langer, 1982).

The molecules of this invention may also be attached to liposomes, which may optionally contain other agents to aid in targeting or administration of the compositions to the desired treatment site. Attachment of the molecules to liposomes may be accomplished by any known cross-linking agent such as heterobifunctional cross-linking agents that have been widely used to couple toxins or chemotherapeutic agents to antibodies for targeted delivery. Conjugation to liposomes can also be accomplished using the carbohydrate-directed cross-linking reagent 4-(4-maleimidophenyl) butyric acid hydrazide (MPBH) (Duzgunes et al., 1992), herein incorporated by reference.

Liposomes containing pharmaceutical molecules may be prepared by well-known methods (See, e.g. DE 3,218,121; Epstein et al., 1985; Hwang et al., 1980; U.S. Pat. Nos. 4,485,045 and 4,544,545). Ordinarily the liposomes are of the small (about 200-800 Angstroms) unilamellar type in which the lipid content is greater than about 30 mol. % cholesterol. The proportion of cholesterol is selected to control the optimal rate of MAG derivative and inhibitor release.

The compositions also will preferably include conventional pharmaceutically acceptable carriers well known in the art (see, e.g., Remington's Pharmaceutical Sciences, 16^{th} Edition, 1980, Mac Publishing Company). Such pharmaceutically acceptable carriers may include other medicinal agents, carriers, genetic carriers, adjuvants, excipients, etc., such as human serum albumin or plasma preparations. The compositions are preferably in the form of a unit dose and will usually be administered one or more times a day.

### 5.0 EXAMPLES

The following examples are provided merely as illustrative of various aspects of the invention and shall not be construed to limit the invention in any way.

A S. cerevisiae YMR325W gene has been discovered as a novel reporter of the sterol biosynthesis pathway in the model organism S. cerevisiae. This invention provides the following examples of the characterization of the S. cerevisiae sterol biosynthesis pathway reporter gene YMR325W described in detail below.

5.1 Genome Reporter Matrix™ Technology

Genome Reporter Matrix™ (GRM) technology was used to generate the gene expression profiles that the sterol biosynthesis inhibition treatments induced in the yeast S. cerevisiae. The GRM was used to generate comprehensive gene expression profiles in the yeast S. cerevisiae. The GRM used in the present invention was a matrix of units comprising living S. cerevisiae cells, the cells in each unit containing one S. cerevisiae reporter fusions (GRM construct) representative of essentially every known gene and hypothetical open reading frame (ORF) of S. cerevisiae. The GRM constructs used in the present invention comprised a promoter, 5′ upstream untranslated region and usually the first four amino acids from one of each hypothetical ORF fused to a gene encoding the green fluorescent protein (GFP). Detailed descriptions of the concept of the GRM can be found in U.S. Pat. Nos. 5,569,588 and 5,777,888, all of which are hereby incorporated by reference, in their entireties. Detailed descriptions of the GRM and how it is used to generate expression profiles can be found in International Publication No. WO/0058521, published Oct. 5, 2000, and in Dimster-Denk, D., et al., 1999, J. of Lipid Research, 40(5):850-860, which are hereby incorporated by reference in their entireties.
5.1.1 Reporter Gene Construct

In a preferred method for constructing the genome reporter constructs used in this invention, a vector comprising a marker gene having an amber mutation and a supF tRNA gene which suppresses the amber mutation is used as the parent vector. A modified version of the methods found in Ashby, M., et al., International Publication WO 00/005821, dated Oct. 5, 2000, which is hereby incorporated by reference in its entirety, is presented below.

A plasmid cloning vector was constructed which comprises a mutant β-lactamase gene with an amber mutation and a supF tRNA gene. Downstream of the supF tRNA gene there is a “stuffer” DNA fragment which is flanked by BsmBI restriction sites. The BsmBI restriction enzyme cuts outside of its six base pair recognition sequence (see, e.g., New England Biolabs 96/97 Catalog, p. 23) and creates a four nucleotide 5′ overhang. When the plasmid cloning vector is digested with BsmBI, the enzyme cleaves within the stuffer DNA and within the adjoining tRNA gene and deletes the four 3′ terminal nucleotides of the gene. The deleted supF tRNA gene encodes a tRNA which cannot fold correctly and is non-functional, i.e., it can not suppress the amber mutation in the mutant β-lactamase gene (β-lactamase (amber)). Downstream from the stuffer DNA fragment is the coding region of a modified green fluorescent protein (“GFP”) gene.

The stuffer DNA was excised from the vector by digestion with BsmBI. The double-stranded DNA at the supF-stuffer fragment junction, produced by BsmBI digestion, is shown below (SEQ ID NO: 4). The tRNA gene sequences are indicated in bold:

5′ . . . supF . . . TC CCCCGGAGACGTC . . . stuffer . . . 3′
3′ . . . AGGGGG CCTCTGCAG . . . 5′
BsmBI

The 3′ terminal sequence of the supF gene necessary for proper function is TCCCCCACCA (SEQ ID NO: 5). The vector, once cleaved with BsmBI, lacks the supF tRNA ACCA terminal nucleotides if the overhangs self-anneal during re-circularization of the plasmid in the absence of insert.

A DNA insert containing the upstream regulatory sequence from a S. cerevisiae ORF was generated as a PCR fragment. Two oligonucleotides were designed to flank the DNA insert sequences of interest on a template DNA and anneal to opposite strands of the template DNA. These oligonucleotides also contained a sequence at their respective 5′ ends that, when converted into a 5′ overhang (in the double-stranded PCR fragment generated using the oligonucleotides), is complementary to the overhangs on the cloning vector generated by BsmBI endonucleolytic cleavage.

Oligonucleotide #1 comprises the 5′ terminal sequence: 5′CCCACCA . . . The remaining nucleotides 3′ to this sequence were designed to anneal to sequences at one end of the DNA insert of choice, in this example, to one of the multitude of S. cerevisiae expression control sequences.

As highlighted in bold above, oligonucleotide #1 comprises the base pairs needed to restore the wild-type 3′ terminal end of the supF tRNA gene. These base pairs are located immediately 3′ to the sequence that allows the insert to anneal to the overhang in the BsmBI-digested pAB4 vector.

Oligonucleotide #2 comprises the 5′ terminal sequence: 5′TCCTG . . . The remaining nucleotides 3′ to this sequence were designed to anneal to sequences at the other end of the DNA insert of choice, in this Example, to one of a variety of S. cerevisiae expression control sequences which may be used according to this invention.

The DNA template (S. cerevisiae genomic DNA) and the two oligonucleotides were annealed and the hybrids were amplified by polymerase chain reaction using Klentaq™ polymerase and PCR buffer according to the manufacturer's instructions (Clontech™). Briefly, 15 ng S. cerevisiae genomic DNA served as template DNA in a 10 μl PCR reaction containing 0.2 mM dNTPs, PCR buffer, Klentaq™ polymerase, and 1 μL of an 8 μM solution containing the primer pairs. The PCR reaction mixture was subjected to the following steps: a) 94° C. for 3 min; b) 94° C. for 15 sec; c) 52° C. for 30 sec; d) 72° C. for 1 min. 45 sec; and e) 4° C. indefinitely. Steps b) through d) were repeated for a total of 30 cycles. The PCR amplification product was purified away from other components of the reaction by standard methods.

To generate the desired 5′ overhangs on the ends of the PCR amplification product, the PCR fragment was treated with DNA polymerase I in the presence of dTTP and dCTP. Under these conditions, DNA polymerase I fills in 3′ overhangs with its 5′ to 3′ polymerase activity and also generates 5′ overhangs with its 3′ to 5′ exonucleolytic activity, which, in the presence of excess dTTP and dCTP, removes nucleotides in a 3′ to 5′ direction until thymidine or a cytosine, respectively, is removed and then replaced.

The overhangs generated by this reaction are:
a) At the 5′ end (supF tRNA restoring end) of the DNA insert:

5′ CCCCACCA . . . becomes 5′ CCCCACCA . . . 3′
3′ GGGGTGGT . . . TGGT . . . 5′

b) At the 3′ end of the DNA insert (joined to the GFP coding sequence)

| | | |
|---|---|---|
| 5′ CAGGA . . . | becomes | 5′ C 3′ |
| 3′ GTCCT . . . | | GTCCT . . . 5′ |

This DNA insert, now comprising 5′ overhangs compatible with one of each of the ends of the BsmBI-cleaved pAB4 vector, was used as substrate in a standard ligation reaction with the BsmBI-cleaved pAB4 vector. The resulting ligation mixture was used to transform competent E. coli cells. The cells were plated on agar plates in the presence of ampicillin.

Colonies that grew in the presence of ampicillin were producing functional β-lactamase enzyme and each harbored the desired recombinant DNA molecule, having a DNA insert with a S. cerevisiae expression control sequence inserted upstream of the modified GFP coding region. The supF gene on vectors which re-ligated without a DNA insert did not express a functional supF tRNA and did not make functional β-lactamase. Thus, they were not found in transformed host cells grown on ampicillin.
5.1.2 Construction of S. cerevisiae Strains

ABY12 (MATa his3Δ1, lev2Δ0, met15Δ0, ura3Δ0) of S. cerevisiae was used. ABY12 is derived from S228C. GRM arrays were grown at 30° C. on solid casamino acid medium (Difco) with 2% glucose and 0.5% Ultrapure Agarose (Gibco BRL). The medium was supplemented with additional amino acids and adenine (Sigma™) at the following concentrations: adenine and tryptophan at 30 μg/ml; histidine, methionine, and tyrosine at 20 μg/ml; leucine and lysine at 40 μg/ml. Stock solutions of the supplements were made at 100× concentrations in water. S. cerevisiae cells were transformed with the reporter plasmids prepared by the method above by electroporation.

5.2 Determining Reporter Gene Expression Levels

Solutions of test compounds were added directly to the solid agar growth media plates prior to addition of S. cerevisiae strains. The individual strains comprising the GRM were maintained as independent colonies (and cultures) in a 96-well format, in medium selecting for the URA3-containing reporter plasmid. Prior to each experiment, fresh dilutions of the reporter-containing strains were inoculated and grown overnight at 30° C. A Hamilton MicroLab 4200, a multichannel gantry robot equipped with a custom pin tool device capable of dispensing 50 nanoliter volumes in a highly reproducible manner, was used to array the matrix of S. cerevisiae strains in a uniform manner onto solid agar growth media at a density of 1536 reporter strains per 110 cm² plate. Fifty nanoliters of S. cerevisiae liquid cultures arrayed onto solid medium by the Hamilton Microlab 4200 results in colony-to-colony signal reproducibility of less than 5% variation. Once arrayed, each plate was grown at 30° C. for 18 hours or at 25° C. for 24 hours.

The level of fluorescence expressed from each reporter gene fusion was determined using a Molecular Dynamics Fluorimager SI. Custom image analysis software was used to quantitate the fluorescence of each colony in the images. Generally, the drug treatments were performed at several concentrations, with the analysis based upon the concentration producing the most informative expression profile.

5.3 Identification of Sterol Biosynthesis Pathway Reporter Genes

Gene expression profiles were generated in the GRM for 36 known inhibitors of sterol biosynthesis (Table 2).

TABLE 2
Known sterol biosynthesis inhibitors profiled in the GRM

Atorvastatin
Bitertanol
Clotrimazole
Cyproconazole
Difenoconazole
Diniconazole
Dodemorph
Dyclonine
Econazole
Fenarimol
Fenbuconazole
Fenpropidin
Fenpropimorph
Fluconazole
Flusilazole
Flutriafol
Fluvastatin
Haloperidol
Itraconazole
Ketoconazole
Lovastatin
Miconazole
Myclobutanil
Nuarimol
Penconazole
Pramoxine
Pravastatin
Prochloraz
Propiconazole
Pyrifenox
Simvastatin
Sulconazole
Terbinafine
Tetraconazole
Triadimenol
Triflumizole

The profiles for these inhibitors are stored in a computerized database as part of a “S. cerevisiae/Genome Reporter Matrix” data set. The data set contained 1,647 expression profiles for approximately 500 unique compounds/molecules and 60 genetic mutants of the S288C strain at the time of analysis. All compound profiles in the data set were generated using the GRM in the same S288C strain background (“Chemical Profiling Strain”). The data set also contained nine profiles generated from two strains harboring mutations in S. Cerevisiae sterol biosynthesis pathway genes Erg11 or Erg13. The mutant phenotypes of these strains were generated by down-regulation of a tetracycline repressible promoter operatively linked to either the Erg11 or Erg13 genes. The expression profiles were collected in these mutant strains by growth on media containing increasing concentrations of tetracycline.

Using a data analysis program, Rosetta Resolver® Gene Expression Data Analysis System (“Resolver® System”) (Agilent Technologies, Palo Alto, Calif.) a two-dimensional agglomerative hierarchical clustering of the 1,647 expression profiles in the “S. cerevisiae/Genome Reporter Matrix” data set was performed. The parameters of the clustering were selected to include only those experiments that had two or more genes up- or down-regulated greater than three-fold and significant at P<=0.01; and only those genes up- or down-regulated at greater than three-fold and at P<=0.01 in two or more experiments. The clustering parameters yielded a hierarchical structure built on 1,533 experiments and 4,337 gene reporters.

Analysis of the hierarchical structure identified a branch of experiments, designated herein as the “sterol branch,” that contained a total of 162 expression profiles, including 138 profiles induced by 36 known inhibitors of sterol biosynthesis (Table 2), 9 profiles induced by two strains harboring mutations in the S. Cerevisiae sterol biosynthesis pathway genes Erg11 and Erg13, and an additional 15 profiles induced by five drugs (benalaxyl, bentiromide, carbetamide indomethacin, and molsidomine) that have undefined modes of action against yeast biology. The gene expression profiles induced by the five drugs indicated that the drugs may have inhibited sterol biosynthesis in yeast under the experimental growth conditions. The 162 experiments/profiles of the sterol branch were used to evaluate the performance of the YMR325W gene reporter in terms of its sensitivity and specificity as an indicator of inhibition of sterol biosynthesis.

Expression of the YMR325W gene reporter was measured in 1,643 out of the 1,647 experiments in the GRM data set. A plot was constructed wherein the ratio (log 10) of YMR325W intensity measured under experimental conditions to intensity measured under control conditions was plotted on the Y-axis, and YMR325W intensity (log 10) measured under experimental conditions was plotted on the X-axis. The plot showed that the YMR325W gene reporter was significantly (p-value<=0.01) up-regulated in one-hundred-thirty-nine of the 1,643 experiments. Using the Resolver® System, data points from the 162 profiles of the sterol branch described supra were “broadcast” or superimposed onto the YMR325W expression plot. The results showed that the vast majority of the data points from the 139 experiments in which the YMR325W gene reporter was significantly up-regulated coincided with data points from the sterol branch. The 1,481 data points that did not coincide with the superimposed data points from the sterol branch were removed from the plot view. The resulting display showed that 125 of the 139 experiments (90% of the experiments) in which the YMR325W gene reporter was significantly (p-value<=0.01) up-regulated were experiments that are part of the sterol branch. The data showed that induction of the up-regulation of the YM325W gene reporter is a highly specific indicator of inhibition of the sterol biosynthesis pathway. Moreover, the YMR325W gene reporter was not up-regulated in only 37 of the 162 sterol branch experiments (23% of the experiments), showing that this reporter is a sensitive indicator of inhibition of the sterol biosynthesis pathway.

In addition, the YMR325W gene reporter was shown to have a good dynamic range, which is an important attribute for use in high throughput screens. The YMR325W gene reporter shows an intensity range across the 1,643 experiments of nearly two logs. The YMR325W gene reporter is an ideal reporter for high throughput screening of chemical libraries or natural products for drug candidates that inhibit the yeast sterol biosynthesis pathway.

5.4 Confirmation of Utility
5.4.1 Liquid High-throughput Like Assay Performance Test

The utility of using the YMR325W gene reporter in a liquid based screen for sterol biosynthesis inhibitors was demonstrated by testing in a 96-well liquid assay.

A non-limiting description of the assay as performed is described below:

1. A YMR325W gene reporter was transformed into wild-type S. Cerevisiae strain ABY12 and grown on solid Casamino Acids media plates.

2. A culture from the solid media plate was used to inoculate a 50 ml liquid culture in Casamino Acids media. The liquid culture was grown overnight at 30° C. to an OD₆₀₀=8, and then was diluted in Casamino Acids media to an OD₆₀₀=1.

3. Drug-like agents Lovastatin, Miconazole, and Fenpropimorph, sterol biosynthesis inhibitors that target different molecular steps in the sterol biosynthesis pathway, and UDP-N-acetyl-glucosamine-1-P transferase (GPT) inhibitor Tunicamycin, a negative control, were used in the assay. Each of the four drugs was added to individual wells (one drug per well) in the second column of a 96-well assay plate and serially diluted (1 to 2) into Casamino Acids media plus 2% DMSO in columns 3-12. The initial concentrations of the drugs were as follows: Lovastatin: 30 ug/ml, Miconazole: 25 ug/ml, Fenpropimorph: 50 ug/ml, and Tunicamycin: 25 ug/ml. The first column of the plate was used as a “no drug” control, containing only Casamino Acids media plus 2% DMSO. Each well contained a final volume of 100 ul of media and/or media plus drug.

4. One-hundred microliters from the liquid reporter strain culture at OD₆₀₀ =1 were added to each well of the 96-well serial dilution plate and mixed. Thus at time zero, the GFP-reporter strain cultures were seeded at a density of OD₆₀₀=0.5. The 96-well assay plate was incubated at 30° C. for about 24 hours.

5. After the 24 hour incubation the 96-well assay plate was imaged in a Molecular Dynamics Fluorimager SI to measure the fluorescence from the YMR325W-GFP reporter.

The YMR325W gene reporter showed increased fluorescence when exposed to the higher concentrations of all three sterol biosynthesis inhibitors tested. No increase in fluorescence was observed for any of the concentrations of the UDP-N-acetyl-glucosamine-1-P transferase (GPT) inhibitor Tunicamycin or in the no drug controls. These results demonstrate the utility of the YMR325W reporter for use as a gene reporter in high-throughput liquid screens for identifying inhibitors of sterol biosynthesis.
5.4.2 Test of YMR325W Reporter in Solid Plate “Halo” Assay

The YMR325W gene reporter was tested in an agar plate “halo” assay to demonstrate the utility of using the YMR325W reporter to identify sterol biosynthesis inhibitors. The assay was designed to mimic assays of natural product producing strains in the presence of ABY12 reporter carrying strains.

A non-limiting description of the assay as performed is described below:

1. A YMR325W reporter was transformed into wild-type S. Cerevisiae strain ABY12 and grown on a solid Casamino Acids media plate.

2. A culture from the solid media plate was used to inoculate a 50 ml liquid culture in Casamino Acids media. The liquid culture was grown overnight at 30° C. to a final OD₆₀₀=8, and then diluted in Casamino Acids media to an OD₆₀₀=1, or about 1×10⁷ cells per milliliter. Five-hundred microliters of cell culture (about 5×10⁶ cells) was spread evenly over the surface of each of two 100 mm solid agar-Casamino-acids-media plates to form a lawn of the YMR325W reporter strain on each plate.

3. Two 6 mm diameter blank paper discs (Becton Dickinson #231039), evenly spaced apart, were placed on top of the agar surface of each plate. On one plate 20 ul of 50 ug/ml of the sterol biosynthesis inhibitor Miconazole was spotted onto one of the two paper discs and 20 ul of 10% DMSO was spotted on the other paper disc as a control. On the other plate 20 ul of 1500 ug/ml Lovastatin was spotted onto one of the two paper discs and 20 ul of 10% DMSO was spotted on the other paper disc as a control. The plates were incubated at 30° C. overnight.

4. After a 72 hour incubation the assay plates were imaged in a Molecular Dynamics Fluorimager SI to measure the fluorescence from the GFP reporters.

Both Miconazole and Lovastatin inhibited the growth of the lawn of the YMR325W reporter strain on each plate as evidenced by the formation of “halo's” of no growth radiating out from the spots where the drug was applied. A zone of high fluorescence at the edge of each halo indicated that the YMR325W reporter was significantly induced by the sterol biosynthesis inhibitors Miconazole and Lovastatin). The Lovastatin induced a much larger zone of fluorescence than the Miconazole. In both instances the DMSO controls caused no zone of growth inhibition and no induction of the YM325W reporter. These results demonstrate the utility of the YMR325W reporter for use in a solid plate assay for discovering agents that block sterol biosynthesis.

The present invention is not be to be limited in scope by the specific embodiments described herein. Indeed, various modifications of the invention in addition to those described herein will become apparent to those skilled in the art from the foregoing description and accompanying drawings. Such modifications are intended to fall within the scope of the appended claims.

Various references are cited herein above, including patent applications, patents, and publications, the disclosures of which are hereby incorporated by reference in their entireties.

1. A method for determining whether a molecule affects the function or activity of a sterol biosynthesis pathway in a S. cerevisiae cell comprising:
(a) contacting said cell with, or recombinantly expressing within said cell, said molecule;
(b) determining whether RNA expression or protein expression in said cell of a target polynucleotide sequence is changed in step (a) relative to the expression of said target polynucleotide sequence in the absence of said molecule, said target polynucleotide being a sequence operatively linked to a promoter native to S. cerevisiae gene YMR325W, or a YMR325W promoter sequence homolog comprising one or more nucleotide substitutions, additions or deletions that do not effect the ability of the sequence to promote transcription of said operatively linked sequence; and
(c) determining that said molecule affects the function or activity of said sterol biosynthesis pathway if expression of said target polynucleotide is changed, or determining that said molecule does not affect the function or activity of said sterol biosynthesis pathway if expression of said target polynucleotide sequence is unchanged.

2. The method of claim 1, wherein said target polynucleotide sequence comprises a marker gene; wherein step (b) comprises determining whether the RNA expression or protein expression of said marker gene is changed in step (a) relative to the expression of said marker gene in the absence of the molecule; and wherein step (c) comprises determining that said molecule affects the function or activity of said sterol biosynthesis pathway if expression of said marker gene is changed, or determining that said molecule does not affect the function or activity of said sterol biosynthesis pathway if expression of said marker gene is unchanged.

3. The method of claim 1 which is a method for determining whether said molecule inhibits sterol biosynthesis such that said cell contacted with the molecule exhibits a lower level of sterol than a second cell which is not contacted with said molecule.

4. The method of claim 1, wherein step (b) comprises determining whether RNA or protein expression of a target polynucleotide sequence regulated by a promoter native to YMR325W is changed.

5. The method of claim 1, wherein step (b) comprises determining whether RNA expression is changed.

6. The method of claim 1, wherein step (b) comprises determining whether protein expression is changed.

7. The method of claim 1 which is a method for determining whether said molecule inhibits sterol biosynthesis, and wherein step (c) comprises determining that said molecule inhibits sterol biosynthesis if expression of said target polynucleotide sequence in step (a) is increased relative to expression of said target polynucleotide sequence in the absence of said molecule.

8. The method of claim 1, wherein the S. cerevisiae cell is a cell that recombinantly expresses said target polynucleotide sequence.

9. The method of claim 1, wherein step (a) comprises contacting said cell with said molecule, and wherein step (a) is carried out in a liquid high throughput-like assay.

10. The method of claim 1, wherein step (a) comprises contacting said cell with said molecule, and wherein step (a) is carried out in a solid plate halo assay.

11. The method of claim 1, wherein step (a) comprises contacting said cell with said molecule, and wherein step (a) is carried out in an agar overlay assay.

12. (canceled)

13. A method for monitoring activity of a sterol biosynthesis pathway in a S. Cerevisiae cell exposed to a molecule comprising:
(a) contacting said cell with, or recombinantly expressing within said cell, said molecule;
(b) determining whether RNA expression or protein expression in said cell of a target polynucleotide sequence is changed in step (a) relative to expression of said target polynucleotide sequence in the absence of said molecule, said target polynucleotide sequence being regulated by a promoter native to a S. cerevisiae YMR325W gene or a YMR325W promoter sequence Homolog-comprising one or more nucleotide substitutions, additions or deletions that do not effect the ability of the sequence to promote regulated transcription of said target polynucleotide sequence; and
(c) determining that the activity of the sterol biosynthesis pathway in said cell is changed if expression of said target polynucleotide is determined to be changed in step (b), or determining that the activity of the sterol biosynthesis pathway in said cell is unchanged if expression of said target polynucleotide is determined to be unchanged in step (b).

14. (canceled)

15. The method of claim 13, wherein step (a) comprises contacting said cell with said molecule.

16. (canceled)

17. The method of claim 13, wherein step (a) comprises recombinantly expressing within said cell said molecule.

18. (canceled)

19. The method of claim 13, wherein step (b) comprises determining that said expression is increased, and step (c) comprises determining that the activity of said sterol biosynthesis pathway is inhibited.

20. The method of claim 13, 15, 17, or 19, wherein said target polynucleotide sequence comprises S. cerevisiae YMR325W.

21. A method for identifying a molecule that modulates expression of a sterol biosynthesis pathway target polynucleotide sequence comprising:
(a) recombinantly expressing in a S. cerevisiae cell, or contacting a S. cerevisiae cell with, at least one candidate molecule; and
(b) measuring RNA or protein expression in said cell of a target polynucleotide sequence, said target polynucleotide sequence being regulated by a promoter native to a S. cerevisiae YMR325W gene or a YMR325W promoter sequence homolog-comprising one or more nucleotide substitutions, additions or deletions that do not effect the ability of the sequence to promote regulated transcription of said target polynucleotide sequence; wherein an increase or decrease in expression of said target polynucleotide sequence relative to expression of said target polynucleotide sequence in the absence of said candidate molecule indicates that said candidate molecule modulates expression of said sterol biosynthesis pathway target polynucleotide sequence.

22. The method of claim 1 wherein said promoter comprises SEQ ID NO: 3 or a SEQ ID NO: 3 homolog-comprising one or more nucleotide Substitutions, additions or deletions that do not effect the ability of the sequence to promote transcription of said operatively linked sequence.

23. The method of claim 2 wherein said marker gene is selected from the group consisting of green fluorescent protein, red fluorescent protein, blue fluorescent protein, luciferase, LEU2, LYS2, ADE2, TRP1, CAN1, CYH2, GUS, CUP1 and chloramphenicol acetyl transferase.

24. (canceled)

25. The method of claim 1, wherein said molecule is selected from the group consisting of natural products, proteins, and small molecules.

26. The method of claim 25, wherein said molecule is purified.

27. The method of claim 25, wherein said molecule is not substantially purified.

28. The method of claim 1, wherein step (a) comprises contacting said cell with a second, test cell, wherein said test cell produces said molecule.

29. The method of claim 28, wherein said molecule is released by said test cell.

30. The method of claim 28, wherein said molecule is secreted by said test cell.
